(19) United States
(12) Patent Application Publication
(10) Pub. No.: US 2005/0119232 A1
(43) Pub. Date: Jun. 2, 2005

(54) Antiparasitic artemisinin derivatives (endoperoxides)

(76) Inventor: Richard K. Haynes, Hong Kong (CN)

Correspondence Address: NORRIS, MCLAUGHLIN & MARCUS, PA, 875 THIRD STREET, 18TH FLOOR, NEW YORK, NY, 10022, US

(21) Appl. No.: 10/506,926

(22) PCT Filed: Feb. 24, 2003
(86) PCT No.: PCT/EP03/01839

(30) Foreign Application Priority Data
Mar. 8, 2002 (EP) 02005233.8

(51) Int. Cl. A61K 31/665; A61K 31/36; C07D 493/14

(52) U.S. Cl. 514/100; 514/452; 549/358

(57) ABSTRACT

This invention relates to certain novel C-10 substituted derivatives of artemisinin, a process for their preparation, their use in the treatnent and/or prophylaxis of diseases caused by infection with a parasite and pharmaceutical compositions containing such C-10 substituted derivatives.

This invention relates to certain novel C-10 substituted derivatives of artemisinin, a process for their preparation, their use in the treatment and/or prophylaxis of diseases caused by infection with a parasite and pharmaceutical compositions containing such C-10 substituted derivatives.

Malaria is the most important human parasitic disease in the world today. Approximately 270 million people throughout the world are infected with malaria, with about 2 million dying each year. The ability of parasites to produce a complex survival mechanism by expressing variant antigens on the surface of infected erythrocytes makes it possible for the parasites to escape from the destructive action of the host immune response against these antigens. In addition, the increasing rate of malaria infection is due to the spread of chloroquine-resistant strains of Plasmodium falciparum and the other multi-drug resistant strains.

In the field of animal health, parasitic diseases are a major problem, especially those diseases which are functionally related to malaria. For instance, neosporosis is a term used to describe diseases caused by parasites of the species Neospora, especially Neospora caninum, in animals. Neospora infections are known to occur in dogs, cattle, sheep, goats and horses.

The final host for Neospora spp., including Neospora caninum, is unknown and, in addition, the complete cycle of development of the parasite is not understood. The asexual phases of reproduction, known as schizogony, and the behaviour of the unicellular tachyzoite/bradyzoite stage have been clarified, however. Tachyzoites are infectious unicellular parasite stages of about 3-7×1-5 mm in size formed after intracellular reproduction termed endodyogeny. Reproduction via tachyzoites takes place preferentially in organelles such as muscle or nerve cells. Pathological symptoms invoked after an infection are associated mainly in those tissues. Some five to six weeks after natural infection in a dog, symptoms of the disease are hypersensitivity caused by inflammation of neuronal cells and increasing tendency to hyperextension of the hind legs. Histopathological lesions are apparent in the nervous system, preferentially in the brain and spinal cord. Extensive non-suppurative inflammations, glial excrescences and perivascular infiltrations of mononuclear cells (macrophages, lymphocytes, plasma cells) dominate, and are also partly apparent in eosinophils and neutrophils. In the muscular system, macroscopically observable necroses and degenerative changes appear. Apart from the more or less strongly developed atrophy, long pale longitudinal stripes are evident.

In California and Australia, Neospora caninum infections appear to be the main cause for abortion in cattle. Symptoms of the disease in cattle are similar to those in the dog. Ataxia is apparent, joint reflexes are weakened and pareses at the hind legs, partly in all four legs, can be observed. The histological picture is similar to that of the dog; mainly non-suppurative meningitis and myelitis.

Data on in vivo activity of compounds suitable against neosporosis are rare because adequate in vivo test systems still have to be developed. Sulfadiazin (administered via drinking water) is effective in experimentally infected mice, only if the treatment was prophylactic, that is, the treatment was started before infection. In dogs, treatment with sulfadiazin and clindamycin is only successful if it is started early, that is, at the appearance of first clinical symptoms as a result of neuronal inflammation.

Coccidiosis, an infection of the small intestine, is relatively rarely diagnosed in humans, where it is caused by Isospora belli. However, humans are also the final host of at least two cyst-forming coccidial species (Sarcocystis suihominis and S. bovihomimis). Consumption of raw or inadequately cooked pork or beef containing such cysts can lead to severe diarrhoea, the cause of which is probably seldom diagnosed correctly. Coccidia (phylum Apicomplexa, suborder Eimeriina) are one of the most successful groups of parasitic protozoans, having conquered virtually every class of Metazoa. The ones that are of particular importance for man are the 60-100 species which parasitise domestic animals and which in some instances can cause very severe losses, especially in poultry, although also in lambs, calves, piglets, rabbits and other animals (see Table A).

TABLE A
Causatives of intestinal coccidiosis in domestic animals

| | number of | |
|---|---|---|
| | Eimeria | |
| | and/or | most pathogenic and/or very |
| | Isospora | common species |
| Animal | species*) | (E = Eimeria, I = Isospora) |
| chicken (Gallus gallus) | 7 | E. tenella, E. necatrix, E. maxima, |
| | | E. acervulina |
| turkey (Meleargidis | 7 | E. meleagrimitis, E. adenoides |
| gallopavo) | | |
| goose (Anser anser) | 6 | E. anseris, E. truncata, E. nocens, |
| | | E. kotlani |
| duck (Anas | 3 | Tyzzeria perniciosa, E. anatis |
| platyhynehus) | | |
| pigeon (Columba livia) | 2 | E. columbarum, E. labbeanea |
| rabbit (Oryctolagus | 11 (12) | E. intestinalis, E. flavescens, |
| cuniculus) | | E. stiedai, E. magna, E. perforans |
| sheep (Ovis arius) | 11 (16) | E. ovinoidalis, E. ashata, E. ovina |
| goat (Capra hircus) | 12 (15) | E. ninakohlyakimovae, E. arloingi |
| cattle (Bos taurus) | 12 (15) | E. zuernii, E. bovis, |
| | | E. auburnensis |
| pig (Sus scofra) | 7 (14) | I. suis, E. debliecki, E. scabra |
| dog (Canis familiaris) | 5 | I. canis, I. (Cystisospora) |
| | | burrowsi |
| cat (Felis catus) | 2 + 6 | I. felis, I. rivolta as final host: |
| | | Sarcocystis bovifelis, S. ovifelis, |
| | | S. fusiformis, S. muris, |
| | | S. cuniculi, Toxoplasma gondii |

*)regarding to Pellerdy (1974), Eckert et al, (1995b, Levine and Ivens (1970) and Mehlhorn 1988)

Most of the pathogenic species are strictly host-specific. They have a complex life cycle with two asexual reproduction phases (schizogony or merogony, and sporogony) and a sexual development phase (gametogony). In view of the major importance of coccidiosis, numerous reviews are available, for instance, by Davies et al. (1963), Hammond and Long (1973), Long (1982, 1990), and Pellerdy (1974). The economically important species sometimes differ very considerably in their sensitivity to medicinal active ingredients. The sensitivity of the different developmental stages to medicinal agents also varies enormously.

As far as the use of drugs is concerned, prophylaxis is the main approach in poultry, in which symptoms do not appear until the phase of increased morbidity, and therapy is the principal strategy in mammals (McDougald 1982). Polyether antibiotics and sulfonamides, among other drugs, are currently used for such treatment and prophylaxis. However, drug-resistant strains of Eimeria have emerged and drug-resistance is now a serious problem. New drugs are therefore urgently required. Given the multiplicity of pathogens and hosts, there is no “ideal model” for identifying and testing anticoccidial agents. For example, most of the many substances used for preventing coccidiosis in poultry are insufficiently effective or even completely ineffective against mamrnalian coccidia (Haberkorn and Mundt; 1989; Haberkorn 1996). Numerous works and sets of instructions have been published on testing of active ingredients in animals for anticoccidial efficacy, for immunisation, etc. One particularly important and comprehensive example is the survey of current methods published by Eckert et al. (1995a).

The compound artemisinin, also known as qinghaosu (1), is a tetracyclic 1,2,4-trioxane occurring in Artemisia annua. Artemisinin and its derivatives dihydroartemisinin (2), artemether (3) and sodium artesunate (4) have been used for the treatment of malaria.

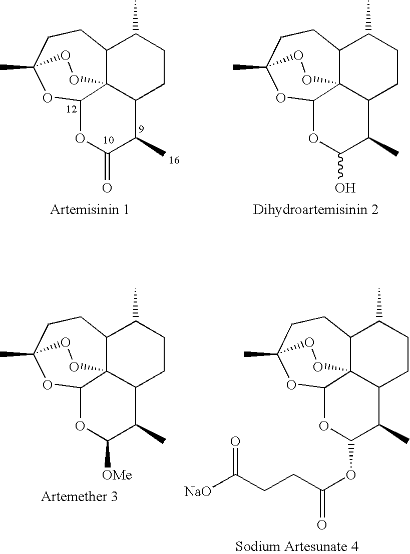

Different modes of action have been proposed by various groups to account for the action of artemisinin and its derivatives in treating malaria (Posner et al., J. Am. Chem. Soc. 1996, 118, 3537,; Posner et al., J. Am. Chem. Soc. 1995, 117, 5885; Posner et al., J. Med. Chem. 1995, 38, 2273). However, irrespective of actual mode of action, all current derivatives suffer from poor oral bioavailability and poor stability (Meshnick et al., Parasitology Today 1996, 12, 79), especially the ‘first generation’ ethers and esters artemether and sodium artesunate obtained from dihydroartemisinin. Extensive chemical studies carried out on artemisinin and derivatives indicate that a cause of instability is the facile opening of the trioxane moiety in artemisinin itself, or in the metabolite common to all currently used derivatives artemether, arteether and artesunate, namely dihydroartemisinin. Ring opening will provide the free hydroperoxide, which is susceptible to reduction. Removal of this group ensures destruction of drug activity with the reduction products being transformed into desoxo metabolites. In order to render ring-opening less facile, the oxygen atom at C-10 can be either removed to provide 10-deoxydihydroartemisinin, or replaced by other groups, and this has provided the basis for the so-called ‘second generation’ compounds which are generally 10-deoxy artemisinin derivatives. In addition, derivatives of artemisinin have also been prepared with a variety of substituents at C-9.

Artemisinin derivatives are also known in which the oxygen atom at C-10 has been replaced by an amine group. For instance, Yang et al (Biorg. Med. Chem. Lett., 1995, 5, 1791-1794) synthesised ten new artemisinin derivatives in which the oxygen atom at C-10 was replaced by a group -NHAr where Ar represents a phenyl, 3-chlorophenyl, 4-chlorophenyl, 3-bromophenyl, 4-bromophenyl, 4-iodophenyl, 4-methylphenyl, 4-methoxyphenyl, 3-carboxylphenyl or 4-carboxylphenyl group. These compounds were tested for in vivo activity against the K173 strain of Plasmodium berghei and found to be active.

WO 00/04024 discloses further C-10 substituted derivatives of artemisinin.

Whilst the current artemisinin derivatives are successful, there are problems associated with stability, bioavailability and potential neurotoxicity. There is also a need for artemisinin derivatives which exhibit a broad spectrum of activity against a variety of parasites.

It has now been discovered that certain C-10 substituted derivatives of artemisinin are effective in the treatment of diseases caused by infection with a parasite. These compounds are particularly effective in the treatment of diseases caused by infection with a parasite of the genera Plasmodium, Neospora or Eimeria, especially Plasmodium falciparum, Neospora caninum and Eimeria tenella which cause malaria, neosporosis and coccidiosis respectively.

According to the present invention there is therefore provided a compound of the general formula I 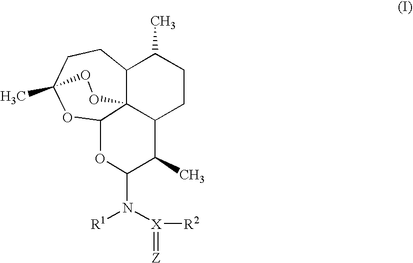
or a salt thereof, or a solvate thereof, or a solvate of a salt thereof,
in which
R¹ represents a hydrogen atom or an optionally substituted alkyl, alkenyl, alkynyl, cycloalkyl, aryl or aralkyl group;
X represents a carbon atom, a sulfur atom, a sulfoxide group S═O or a group PR³, P—O—R³ or P—N(R⁴)—R³ where R³ and R⁴ each independently represent a hydrogen atom or an optionally substituted alkyl, alkenyl, alkynyl, cycloalkyl, aryl or aralkyl group;
Z represents an oxygen atom, a sulfur atom or a group NR⁵ where R⁵ represents a hydrogen atom or an optionally substituted alkyl, alkenyl, alkynyl, cycloalkyl, aryl or aralkyl group; and
R² represents a hydrogen atom or an optionally substituted alkyl, alkenyl, alkynyl, cycloalkyl, aryl or aralkyl group, or a group N(R⁶)₂, NHNH₂, NR⁶NHR⁶ or NR⁶N(R⁶)₂, or a group OR⁶ or SR⁶ where each R⁶ independently represents a hydrogen atom or an optionally substituted alkyl, alkenyl, alkynyl, cycloalkyl, aryl or aralkyl group, or a 10α-dihydroartemisinyl group, or R² represents a group OR⁷ or NR⁶R⁷ where R⁶ represents a group as defined above and R⁷ represents a bond attached as a substituent to R⁵ together with the interjacent group —X═Z— forming an optionally substituted heterocyclic group where Z represents a group NR⁵, or R⁷ represents a bond attached as a substituent to R¹ together with the interjacent group —N—X(═Z)— forming an optionally substituted heterocyclic group.

Suitable salts include acid addition salts and these may be formned by reaction of a suitable compound of formula I with a suitable acid, such as an organic acid or a mineral acid. Acid addition salts formed by reaction with a mineral acid are particularly preferred, especially salts formed by reaction with hydrochloric or hydrobromic acid.

A solvate according to the present invention is any such form of the compounds forming a complex in solid or liquid state by coordination with solvent molecules. Hydrates are a special form of solvates formed by coordination with water molecules.

Any alkyl, alkenyl or alkynyl group, unless otherwise specified, may be linear or branched and may contain up to 12, preferably up to 6, and especially up to 4 carbon atoms. Preferred alkyl groups are methyl, ethyl, propyl and butyl. It is preferred that any alkenyl or alkynyl group is not an alk-1-enyl or alk-1-ynyl group. In other words, there should preferably be at least one methylene group —CH₂— or similar sp³-hybridised center between a carbon atom forming part of the double or triple C—C bond and the nitrogen atom to which the group is attached. Preferred alkenyl and alkynyl groups include propenyl, butenyl, propynyl and butynyl groups. When an alkyl moiety forms part of another group, for example the alkyl moiety of an aralkyl group, it is preferred that it contains up to 6, especially up to 4, carbon atoms. Preferred alkyl moieties are methyl and ethyl.

An aryl group may be any aromatic hydrocarbon group and may contain from 6 to 24, preferably 6 to 18, more preferably 6 to 16, and especially 6 to 14, carbon atoms. Preferred aryl groups include phenyl, naphthyl, anthryl, phenanthryl and pyryl groups, especially a phenyl or naphthyl, and particularly a phenyl, group. When an aryl moiety forms part of another group, for example the aryl moiety of an aralkyl group, it is preferred that it is a phenyl, naphthyl, anthryl, phenanthryl or pyryl, especially phenyl or naphthyl, and particularly a phenyl, moiety.

An aralkyl group may be any alkyl group substituted by an aryl group. A preferred aralkyl group contains from 7 to 30, particularly 7 to 24 and especially 7 to 18, carbon atoms, particularly preferred aralkyl groups being benzyl, naphthylmethyl, anthrylmethyl, phenanthrylmethyl and pyrylmethyl groups. A particularly preferred aralkyl group is a benzyl group.

A cycloalkyl group may be any saturated cyclic hydrocarbon group and may contain from 3 to 12, preferably 3 to 8, and especially 3 to 6, carbon atoms. Preferred cycloalkyl groups are cyclopropyl, cyclopentyl and cyclohexyl groups.

A heteroaryl group may be any aromatic monocyclic or polycyclic ring system which contains at least one heteroatom. Preferably, a heteroaryl group is a 5- to 18-membered, particularly a 5- to 14-membered, and especially a 5- to 10-membered, aromatic ring system containing at least one heteroatom selected from oxygen, sulphur and nitrogen atoms. Preferred heteroaryl groups include pyridyl, pyrylium, thiopyrylium, pyrrolyl, furyl, thienyl, indolinyl, isoindolinyl, indolizinyl, imidazolyl, pyridonyl, pyronyl, pyrimidinyl, pyrazinyl, oxazolyl, thiazolyl, purinyl, quinolinyl, isoquinolinyl, quinoxalinyl, pyridazinyl, benzofuranyl, benzoxazolyl and acridinyl groups. A C-linked heteroaryl group is therefore a heteroaryl group as defined above which is linked to the tetracyclic 1,2,4-trioxane moiety of a compound of general formula I via a carbon atom in the heteroaromatic ring system.

A heterocyclic group may be any monocyclic or polycyclic ring system which contains at least one heteroatom and may be unsaturated or partially or fully saturated. The term “heterocyclic” thus includes heteroaryl groups as defined above as well as non-aromatic heterocyclic groups. Preferably, a heterocyclic group is a 3- to 18-membered, particularly a 3- to 14-membered, especially a 5- to 10-membered, ring system containing at least one heteroatom selected from oxygen, sulphur and nitrogen atoms. Preferred heterocyclic groups include the specific heteroaryl groups named above as well as pyranyl, piperidinyl, pyrrolidinyl, dioxanyl, piperazinyl, morpholinyl, thiomorpholinyl, morpholinosulphonyl, tetrahydroisoquinolinyl and tetrahydrofuranyl groups.

A heterocyclylalkyl group may be any alkyl group substituted by a heterocyclic group. Preferably, the heterocyclic moiety is a 3- to 18-membered, particularly a 3- to 14-membered, and especially a 5- to 10-membered, heterocyclic group as defined above and the alkyl moiety is a C₁₋₆ alkyl, preferably C1-4 alkyl, and especially methyl, group.

An amino acid may be any α-amino acid, such as glycine, alanine, valine, leucine, isoleucine, serine, threonine, cysteine, cystine, methionine, aspartic acid, glutamic acid, aspargine, glutamine, lysine, hydroxylysine, arginine, histidine, phenylalanine, tyrosine, tryptophan, proline, hydroxyproline or phenylglycine, and includes both D-and L-configurations. An amino acid ester may be any ester of such an amino acid, alkyl esters, particularly C₁₋₄ alkyl esters, being especially preferred.

When any of the foregoing substituents are designated as being optionally substituted, the substituent groups which are optionally present may be any one or more of those customarily employed in the development of pharmaceutical compounds and/or the modification of such compounds to influence their structure/activity, stability, bioavailability or other property. Specific examples of such substituents include, for example, halogen atoms, nitro, cyano, hydroxyl, cycloalkyl, alkyl, alkenyl, haloalkyl, alkoxy, haloalkoxy, amino, alkylamino, dialkylamino, formyl, alkoxycarbonyl, carboxyl, alkanoyl, alkylthio, alkylsulphinyl, alkylsulphonyl, alkylsulphonato, arylsulphinyl, arylsulphonyl, arylsulphonato, carbamoyl, alkylamido, aryl, heterocyclic and alkyl- or aryl-substituted heterocyclic groups. When any of the foregoing substituents represents or contains an alkyl or alkenyl substituent group, this may be linear or branched and may contain up to 12, preferably up to 6, and especially up to 4, carbon atoms. A cycloalkyl group may contain from 3 to 8, preferably from 3 to 6, carbon atoms. An aryl group or moiety may contain from 6 to 10 carbon atoms, phenyl groups being especially preferred. A heterocyclic group or moiety may be a 5- to 10-membered ring system as defined above. A halogen atom may be a fluorine, chlorine, bromine or iodine atom and any group which contains a halo moiety, such as a haloalkyl group, may thus contain any one or more of these halogen atoms.

In one aspect, it is preferred, that R¹ represents a hydrogen atom, a methyl group, ethyl group or longer chain alkyl group or a branched alkyl group containing up to 9 carbon atoms, preferably a hydrogen atom, a methyl group or an ethyl group.

In another preferred aspect, X represents a carbon atom, a sulfur atom, or a group PR³, P—O—R³ or P—N(R⁴)—R³ where R³ and R⁴ each independently represent a C₆₋₁₈ aryl group or a 5- to 10-membered C-linked heteroaryl group or a 5- to 10-membered heterocyclyl-C₁₋₆ alkyl group optionally substituted by one or more substituents selected from the group consisting of halogen atoms, hydroxyl, C₁₋₄ alkyl, C₂₋₄ alkenyl, C₁₋₄ haloalkyl, C₁₋₄ alkoxy, C₁₋₄ haloalkoxy, amino, C₁₋₄ alkylamino, di(C₁₋₄ alkyl)amino and carboxyl groups. Preferably, X represents a carbon atom or a sulfur atom.

In a further preferred aspect, Z represents an oxygen atom, or a group NR⁵ where R⁵ represents a hydrogen atom, a methyl group, ethyl group or longer chain alkyl group or branched alkyl group containing up to 9 carbon atoms, or a C₆₋₁₈ aryl group or a 5- to 10-membered C-linked heteroaryl group or a 5- to 10-membered heterocyclyl-C₁₋₆ alkyl group optionally substituted by one or more substituents selected from the group consisting of halogen atoms, hydroxyl, C₁₋₄ alkyl, C₂₋₄ alkenyl, C1-4 haloalkyl, C₁₋₄ alkoxy, C₁₋₄ haloalkoxy, amino, C₁₋₄ alkylamino, di(C₁₋₄ alkyl)amino and carboxyl groups.

In another preferred aspect, R² represents a hydrogen atom or an optionally substituted alkyl, alkenyl, alkynyl, cycloalkyl, aryl or aralkyl group, or a group OR⁶, SR⁶, NH₂, NHR⁶, or N(R⁶)₂ where each R⁶ indepently represents a methyl group, ethyl group or longer chain alkyl group or branched alkyl group containing up to 9 carbon atoms atoms, or is a C₆₋₁₈ aryl group or a 5- to 10-membered C-linked heteroaryl group or a 5- to 10-membered heterocyclyl-C₁₋₆ alkyl group optionally substituted by one or more substituents selected from the group consisting of halogen atoms, hydroxyl, C₁₋₄ alkyl, C₂₋₄ alkenyl, C₁₋₄ haloalkyl, C₁₋₄ alkoxy, C₁₋₄ haloalkoxy, amino, C₁₋₄ alkylamino, di(C₁₋₄ alkyl)amino and carboxyl groups. Preferably, R² represents a group NH₂, or a group NHR⁶ where R⁶ represents an alkyl or aryl group, or a group N(R⁶)₂ where R⁶ represent identical or differentiated alyl groups.

In a further preferred aspect, R¹ represents a hydrogen atom or an optionally substituted allyl, alkenyl, alkynyl, cycloalkyl, aryl or aralkyl group, preferably a hydrogen atom or an alkyl group, more preferably a hydrogen atom or a methyl group or an ethyl group; X represents a carbon, phosphorus or sulfur atom, preferably a carbon or sulfuir atom; Z represents an oxygen atom or a group NR⁵ in where R⁵ represents a hydrogen atom or an optionally substituted alkyl, alkenyl, alkynyl, cycloalkyl, aryl or aralkyl group, preferably an oxygen atom; and R² represents a group OR⁶, SR⁶, NH₂, NHR⁶, or N(R⁶)₂ where each R⁶ independently represents a hydrogen atom or an optionally substituted alkyl, alkenyl, alkynyl, cycloalkyl, aryl or aralkyl group, or a 10α-dihydroartemisinyl group, preferably a hydrogen atom or an optionally substituted alkyl or aryl group, more preferably R² represents a group NH₂, or a group NHR⁶ where R⁶ represents an alkyl group, or a group N(R⁶)₂ where R⁶ represent identical or differentiated alkyl groups.

In an especially preferred aspect, R¹ represents a hydrogen atom, X represents a sulfoxide group S═O, Z represents an oxygen atom, and R² represents a group NH₂; or R¹ represents a hydrogen atom, X represents a carbon atom, Z represents a group NH, and R² represents a group NHR⁶ where R⁶ represents a hydrogen atom or an optionally substituted alkyl, cycloalkyl, aryl or aralkyl group; or R¹ represents a hydrogen atom, X represents a carbon atom, Z represents an oxygen atom, and R² represents a group NHR⁶ where R⁶ is a hydrogen atom or an optionally substituted alkyl, cycloalkyl, aryl or aralkyl group.

It should also be appreciated that the compounds of general formula I are capable of existing as different geometric and optical isomers. The present invention thus includes both the individual isomers and mixtures of such isomers.

The present invention includes a compound of the general formula I as defined above for use in the treatment and/or prophylaxis of a disease. Preferably, the disease is a disease caused by infection with a parasite. More preferably, the disease is a disease caused by infection with a parasite of the genus Plasmodium, the genus Neospora, or the genus Eimeria.

The present invention also provides the use of a compound of the general formula I as defined above for the manufacture of a medicament for the treatment and/or prophylaxis of a disease caused by infection with a parasite. Preferably, the parasite is an organism of the genus Plasmodium, the genus Neospora, or the genus Eimeria.

The present invention also provides a process for the preparation of a compound of the general formula I which comprises reacting a compound of the general formula II 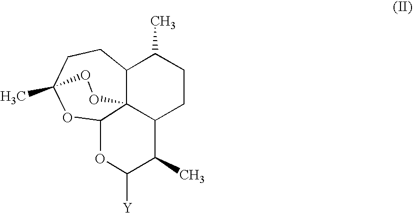
in which Y represents a group containing an oxygen atom attached to the carbon atom of the artemisinin nucleus and also to a hydrogen atom or trimethylsilyl group, with a suitable halogenating agent to form a compound of the general formula II in which Y represents a halogen atom; and, if desired, reacting the compound of general formula II thus formed with an amine of the general formula R¹NHX(═Z)R² where R¹, R², X and Z are as defined above to form a compound of general formula I.

Suitable halogenating agents for forming compounds of the general formula in which Y represents a halogen atom include diethylaminosulphur trifluoride, chlorotrimethylsilane, bromotrimethylsilane and iodotrimethylsilane. In particular, compounds of the general formula II in which Y represents a chlorine, bromine or iodine atom may be prepared by reacting a compound of the general formula II in which Y represents a trimethylsilyloxy group with a suitable chlorinating, brominating or iodinating agent respectively, such as chlorotrimethlysilane, bromotrimethylsilane or iodotrimethylsilane respectively. This reaction may be conveniently carried out in the presence of a solvent. Suitable solvents include aromatic solvents such as toluene, or halogenated hydrocarbons, especially chlorinated hydrocarbons, such as dichloromethane. Preferably, the reaction is carried out at a temperature of −30 to +20° C., particularly −5 to +10° C., about 0° C. being especially preferred.

Compounds of the general formula II in which Y represents a fluorine atom may be conveniently prepared by reacting a compound of the general formula II in which Y represents a hydroxyl group with a suitable fluorinating agent, such as diethylaminosulphur trifluoride. This reaction may be conveniently carried out in the presence of a solvent, suitable solvents including aromatic solvents such as toluene and halogenated hydrocarbons, especially chlorinated hydrocarbons, such as dichloromethane. Preferably, the reaction is carried out at −5° C. to room temperature, that is, −5 to +35° C., preferably 0 to 30° C. The reaction may also be carried out under an inert atmosphere, such as nitrogen.

The reaction of an amine of the type R¹NHX(═Z)R² where R¹, R², X and Z are as defined above with a compound of the general formula II in which Y represents a halogen, preferably a chlorine or bromine, atom to form a compound of the general formula II in which Y represents the group R¹NX(═Z)R² where R¹, R², X and Z are as defined above may be conveniently carried out in the presence of a solvent. Suitable solvents include halogenated hydrocarbons, especially chlorinated hydrocarbons, such as dichloromethane, and ethers, such as tetrahydrofuran. Preferably, the reaction is carried out at a temperature of −5 to +5° C., 0° C. being especially preferred.

When a compound of the general formula II in which Y represents a bromine atom is to be further reacted with an amine to form a compound of the general formula II in which Y represents a group R¹NHX(═Z)R² where R¹, R², X and Z are as defined above, it is preferred that the compound of the general formula II in which Y represents a bromine atom is generated in situ by reacting a compound of the general formula II in which Y represents a trimethylsiloxy group with bromotrimethylsilane.

A compound of the general formula II in which Y represents a trimethylsiloxy group may be prepared by reacting dihydroartemisinin, that is, the compound of general formula II in which Y represents a hydroxyl group, with chlorotrimethylsilane in the presence of a base, such as pyridine or triethylamine. Preferably, the reaction is carried out at room temperature, that is, 15 to 35° C., preferably 20 to 30° C.

Dihydroartemisinin, that is, the compound of general formula II in which Y represents a hydroxyl group, is a known compound and can be prepared by known processes.

The present invention also provides a pharmaceutical composition which comprises a carrier and, as active ingredient, a compound of the general formula I as defined above.

A pharmaceutically acceptable carrier may be any material with which the active ingredient is formulated to facilitate administration. A carrier may be a solid or a liquid, including a material which is normally gaseous but which has been compressed to form a liquid, and any of the carriers normally used in formulating pharmaceutical compositions may be used. Preferably, compositions according to the present invention contain 0.5 to 95% by weight of active ingredient.

The compounds of general formula I can be formulated as, for example, tablets, capsules, suppositories or solutions. These formulations can be produced by known methods using conventional solid carriers such as, for example, lactose, starch or talcum or liquid carriers such as, for example, water, fatty oils or liquid paraffins. Other carriers which may be used include materials derived from animal or vegetable proteins, such as the gelatins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; polypeptide/protein or polysaccharide complexes such as gelatin-acacia complexes; sugars such as mannitol, dextrose, galactose and trehalose; cyclic sugars such as cyclodextrin; inorganic salts such as sodium phosphate, sodium chloride and aluminium silicates; and amino acids having from 2 to 12 carbon atoms such as a glycine, L-alanine, L-aspartic acid, L-glutamic acid, L-hydroxyproline, L-isoleucine, L-leucine and L-phenylalanine.

Auxiliary components such as tablet disintegrants, solubilisers, preservatives, antioxidants, surfactants, viscosity enhancers, colouring agents, flavouring agents, pH modifiers, sweeteners or taste-masking agents may also be incorporated into the composition. Suitable colouring agents include red, black and yellow iron oxides and FD & C dyes such as FD & C blue No. 2 and FD & C red No. 40 available from Ellis & Everard. Suitable flavouring agents include mint, raspberry, liquorice, orange, lemon, grapefruit, caramel, vanilla, cherry and grape flavours and combinations of these. Suitable pH modifiers include citric acid, tartaric acid, phosphoric acid, hydrochloric acid and maleic acid. Suitable sweeteners include aspartame, acesulfame K and thaumatin. Suitable taste-masking agents include sodium bicarbonate, ion-exchange resins, cyclodextrin inclusion compounds, adsorbates or microencapsulated actives.

For treatment of and prophylaxis against coccidiosis and related parasites, for instance, in poultry, especially in chickens, ducks, geese and turkeys, 0.1 to 100 ppm, preferably 0.5 to 100 ppm of the active compound may be mixed into an appropriate, edible material, such as nutritious food. If desired, the amounts applied can be increased, especially if the active compound is well tolerated by the recipient. Accordingly, the active compound can be applied with the drinking water.

For the treatment of a single animal, for instance, for the treatment of coccidiosis in mammals or toxoplasmosis, amounts of 0.5 to 100 mg/kg body weight active compound are preferably administered daily to obtain the desired results. Nevertheless, it may be necessary from time to time to depart from the amounts mentioned above, depending on the body weight of the experimental animal, the method of application, the animal species and its individual reaction to the drug or the kind of formulation or the time or interval in which the drug is applied. In special cases, it may be sufficient to use less than the minimum amount given above, whilst in other cases the maximum dose may have to be exceeded. For a larger dose, it may be advisable to divide the dose into several smaller single doses.

The present invention also includes a pharmaceutical composition as described above for use in the treatment and/or prophylaxis of a disease caused by infection with a parasite. Preferably, the parasite is an organism of the genus Plasmodium, the genus Neospora, or the genus Eimeria.

The present invention also provides a method for treating a disease caused by infection with a parasite which comprises administering to a host in need of such treatment a therapeutically effective amount of a compound of the general formula I as defined above. Preferably, the parasite is an organism of the genus Plasmodium, the genus Neospora, or the genus Eimeria.

The present invention is further illustrated by the following examples.

### EXAMPLES

### Example 1

### 10α-(Sulfamino)dihydroartemisinin

(Formula I: R¹═H; X S═O; Z=O; R²═NH₂)

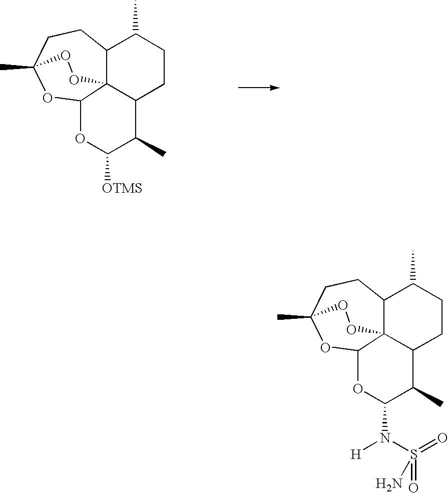

Trimethylsilyl bromide (0.16 g, 0.14 ml, 1.05 mmol) was added dropwise to a cold (0° C.) stirred solution of 10α-(trimethylsiloxy)dihydroartemisinin (356 mg, 1.0 mmol) in dichloromethane (5 ml). After 15 min. (tlc), a solution of sulfamide (0.19 g, 2.0 inmol) in THF (6 ml) was added. After 1.5 h, the reaction was quenched with saturated NaHCO_{3(aq)} (10 ml) and extracted with diethyl ether (3×10 ml). The organic extracts were combined and dried (MgSO₄). Filtration and evaporation of filtrate gave a dark green solid which was purified by column chromatography on silica with ethyl acetate-hexanes (40:60) as eluent. Pooling and evaporation of appropriate fractions gave a white powder (211.48 mg, 57%). M.p. 168-168.7° C. (decomposed); [α]_{D}²²+16.76° (c 0.68 MeOH); IR (KBr) νₘₐₓ 3387, 3226, 2959, 2934, 2880, 1631, 1456, 1375, 1323, 1308, 1147, 1128, 1024; ¹H-NMR: δ_{H} 7.79 (1H, d, NH, J=8.63 Hz), 6.44 (2H, s, NH₂), 5.37 (1H, s, H-12), 4.58 (1H, pseudo-triplet, H-10, J=9.23 Hz), 2.31-2.12 (2H, m), 2.00-1.96 (1H, m), 1.82-1.77 (1H, m), 1.64-1.61 (2H, m), 1.51-1.31 (4H, m), 1.28 (3H,s 3-Me), 1.20-1.12 (1H, m), 1.10-0.94 (1H, m), 0.89 (3H, d, 9-Me, J=6.23 Hz), 0.78 (3H, d, 6-Me, J=7.11 Hz; ¹³C-NMR: δ_{c}103.31, 90.61, 80.47, 79.98, 51.36, 45.14, 36.34, 36.01, 33.66, 31.56 25.66, 24.57, 21.24, 20.46, 13.60MS (CI, CH₄) m/z 363 (MH⁺, 7%), 364 (MH⁺, ¹³C, 1%); Analysis calculated for C₁₅H₂₆N₂O₆S requires C, 49.71; H, 7.23; found C, 49.59; H, 7.29; N, 7.58

### Example 2

### Bis[(10α-dihydroartemisinyl)]sulfamide

(Formula I: R¹═H; X═SO; Z═O; R²═NH(10α-dihydroartemisinyl))

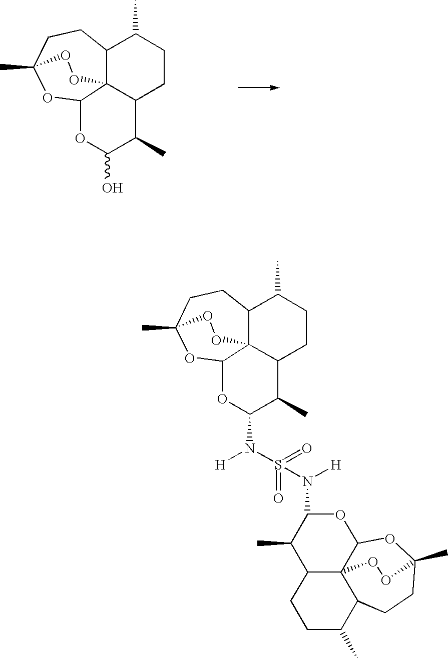

Trimethylsilyl chloride (0.42 g, 0.49 ml, 3.87 mmol) was added to a cold (0° C.) stirred mixture of dihydroartemisinin (0.5 g, 1.76 inmol) and sodium bromide (199 mg, 1.94 mmol) in toluene (2 ml). After 1 h (tlc), a solution of sulfamide (85 mg, 0.88 mmol) in THF (2 ml) was added rapidly. After 3.5 h, water (5 ml) followed by diethyl ether (10 ml) were added. The aqueous layer was separated and extracted further with diethly ether (3×5 ml). The organic extracts were combined and dried (MgSO4). Filtration and evaporation of filtrate gave a dark green glassy solid which was purified by column chromatography on silica using ethyl acetate-hexanes (25:75) as eluent. Pooling and evaporation of appropriate fractions gave a pale yellow powder (178.9 mg, 32%). M.p. 183-184° C. (decomposed); IR (KBr) νₘₐₓ 3035, 3216, 2927, 2875, 1458, 1381, 1348, 1167, 1149, 1130, 1113, 1024, 916, 876, 735; ¹H-NMR: δ_{H} 5.42 (2H, broad-doublet, 2×NH, J=11.2 Hz), 5.35 (2H, s, 2×H-12), 4.81 (2H, pseudo-triplet, 2×H-10, J=10.4 Hz), 2.36-2.29 (4H, m), 2.03-2.00 (2H, m), 1.89-1.86 (2H, m), 1.76-1.70 (4H, m), 1.57-1.54 (2H, m), 1.45 (6H, s, 2×3-Me), 1.44-b 1.42 (2H, m), 1.38-1.77 (6H, m), 1.02-0.97 (2H, m), 0.95(6H, d, 2×9-Me, J=6.4 Hz), 0.91 (6H, d, 2×6-Me, J=6.8 Hz); ¹³C-NMR: δ_{c} 104.32, 91.06, 82.80, 79.50, 51.68, 45.67, 37.30, 36.31, 34.09, 32.46, 25.71, 24.80, 21.55, 20.28, 13.42,

1. A compound of the formula I: 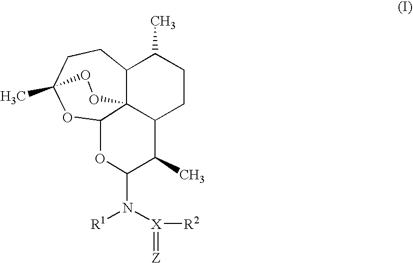
or a salt thereof, or a solvate thereof, or a solvate of a salt thereof,
in which
R¹ represents a hydrogen atom or an optionally substituted alkyl, alkenyl, alkynyl, cycloalkyl, aryl or aralkyl group;
X represents a carbon atom, a sulfur atom, a sulfoxide group S═O or a group PR³, P—O—R³ or P—N(R⁴)—R³ where R³ and R⁴ each independently represent a hydrogen atom or an optionally substituted alkyl, alkenyl, alkynyl, cycloalkyl, aryl or aralkyl group;
Z represents an oxygen atom, a sulfur atom or a group NR⁵ where R⁵ represents a hydrogen atom or an optionally substituted alkyl, alkenyl, alkynyl, cycloalkyl, aryl or aralkyl group; and
R² represents a hydrogen atom or an optionally substituted alkyl, alkenyl, alkynyl, cycloalkyl, aryl or aralky group, or a group N(R⁶)₂, NHNH₂, NR⁶NHR⁶ or NR⁶N(R⁶)₂, or a group OR⁶ or SR⁶ where each R⁶ independently represents a hydrogen atom or an optionally substituted alkyl, alkenyl, alkynyl, cycloalkyl, aryl or aralkyl group, or a 10α-dihydroartemisinyl group, or R² represents a group OR⁷ or NR⁶R⁷ where R⁶ represents a group as defined above and R⁷ represents a bond attached as a substituent to R⁵ together with the group —X═Z- forming an optionally substituted heterocyclic group where Z represents a group NR⁵, or R⁷ represents a bond attached as a substituent to R¹ together with the group —N—X(═Z)- forming an optionally substituted heterocyclic group.

2. A compound according to claim 1 in which R¹ represents a hydrogen atom, a methyl group, ethyl group or longer straight-chain alkyl group or a branched alkyl group containing up to 9 carbon atoms.

3. A compound according to claim 1 in which X represents a carbon atom, a sulfur atom, or a group PR³, P—O—R³ or P—N(R⁴)—R³ where R³ and R⁴ each independently represent a C₆₋₁₈ aryl group or a 5- to 10-membered C-linked heteroaryl group or a 5- to 10-membered heterocyclyl-C₁₋₆ alkyl group optionally substituted by one or more substituents selected from the group consisting of halogen atoms, hydroxyl, C₁₋₄ alkyl, C₂₋₄ alkenyl, C₁₋₄ haloalkyl, C₁₋₄ alkoxy, C₁₋₄ haloalkoxy, amino, C₁₋₄ alkylamino, di(C₁₋₄ alkyl)amino and carboxyl groups.

4. A compound according to claim 1 in which Z represents an oxygen atom, or a group NR⁵ where R⁵ represents a hydrogen atom, a methyl group, ethyl group or longer straight-chain alkyl group or branched alkyl group containing up to 9 carbon atoms or a C₆₋₁₈ aryl group or a 5- to 10-membered C-linked heteroaryl group or a 5- to 10-membered heterocyclyl-C₁₋₆ alkyl group optionally substituted by one or more substituents selected from the group consisting of halogen atoms, hydroxyl, C₁₋₄ alkyl, C₂₋₄, alkenyl, C₁₋₄ haloalkyl, C₁₋₄ alkoxy, C₁₋₄ haloalkoxy, amino, C₁₋₄ alkylamino, di(C₁₋₄ alkyl)amino and carboxyl groups.

5. A compound according to claim 1 in which R² represents a hydrogen atom or an optionally substituted alkyl, alkenyl, alkynyl, cycloalkyl, aryl or aralkyl group, or a group OR⁶, SR⁶, NH₂, NHR⁶, or N(R⁶)₂ where each R⁶ independently represents a methyl group, ethyl group or longer straight-chain alkyl group or branched alkyl group containing up to 9 carbon atoms, or is a C₆₋₁₈ aryl group or a 5- to 10-membered C-linked heteroaryl group or a 5- to 10-membered heterocyclyl-C₁₋₆ alkyl group optionally substituted by one or more substituents selected from the group consisting of halogen atoms, hydroxyl, C₁₋₄ alkyl, C₂₋₄ alkenyl, C₁₋₄ haloalkyl, C₁₋₄ alkoxy, C₁₋₄ haloalkoxy, amino, C₁₋₄ alkylamino, di(C₁₋₄ alkyl)amino and carboxyl groups.

6. A compound according to claim 1 in which R¹ represents a hydrogen atom or an optionally substituted alkyl, alkenyl, alkynyl, cycloalkyl, aryl or aralkyl group; X represents a carbon, phosphorus or sulfur atom; Z represents an oxygen atom or a group NR⁵ in where R⁵ represents a hydrogen atom or an optionally substituted alkyl, alkenyl, alkynyl, cycloalkyl, aryl or aralkyl group; and R² represents a group OR⁶, SR⁶, NH₂, NHR⁶, or NH², or N(R⁶)₂ where each R⁶ independently represents a hydrogen atom or an optionally substituted alkyl, alkenyl, alkynyl, cycloalkyl, aryl or aralkyl group, or a 10α-dihydroartemisinyl group.

7. A compound according to claim 1 in which R¹ represents a hydrogen atom, X represents a sulfoxide group S═O, Z represents an oxygen atom, and R² represents a group NH₂, or in which R¹ represents a hydrogen atom, X represents a carbon atom, Z represents a group NH, and R² represents a group NHR⁶ where R⁶ represents a hydrogen atom or an optionally substituted alkyl, cycloalkyl, aryl or aralkyl group; or in which R¹ represents a hydrogen atom, X represents a carbon atom, Z represents an oxygen atom, and R² represents a group NHR⁶ where R⁶ is a hydrogen atom or an optionally substituted alkyl, cycloalkyl, aryl or aralkyl group.

8. A process for the preparation of a compound of the general formula I according to claim 1 which comprises reacting a compound of the formula II comprising an atremisinin nucleus: 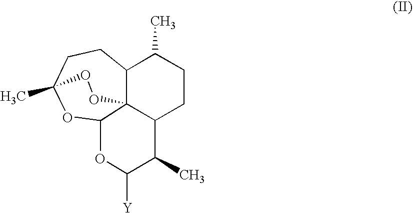
in which Y represents a group comprising an oxygen atom attached to the carbon atom of the artemisinin nucleus and also to a hydrogen atom or trimethylsiyl group, with a suitable halogenating agent to form a compound of the formula II in which Y represents a halogen atom; and, if desired, reacting the compound of formula II in which Y represents a halogen atom with an amine of the formula:
R¹NHX(═Z)R²
where R¹, R², X and Z are as defined in claim 1 to form a compound of the formula I.

9. (canceled.)

10. A pharmaceutical composition which comprises a carrier and a therapeutically effective amount of a compound according to claim 1.

11. (canceled.)

12. (canceled.)

13. A method for treating a disease caused by infection with a parasite which comprises administering to a host in need of such treatment a therapeutically effective amount of a compound according to claim 1.

14. A compound according to claim 2 in which R¹ represents a hydrogen atom, a methyl group or an ethyl group.

15. A compound according to claim 6 in which R¹ represents a hydrogen atom or an alkyl group; X represents a carbon or sulfur atom; Z represents an oxygen atom; R6 represents a hydrogen atom or an optionally substituted alkyl or aryl group; or R² represents a group NH₂, or a group NHR⁶ where R⁶ represents an alkyl group, or a group N(R⁶)₂ where R⁶ represents identical or different alkyl groups.

16. A compound according to claim 15 in which R¹ represents a hydrogen atom or a methyl group or an ethyl group; or R² represents a group NH₂, or a group NHR⁶ where R⁶ represents an alkyl group, or a group N(R⁶)₂ where R⁶ represents identical or different alkyl groups.
